(19) United States
(12) Patent Application Publication
(10) Pub. No.: US 2016/0175245 A1
(43) Pub. Date: Jun. 23, 2016

(54) TREATMENT OF DISEASES ASSOCIATED WITH MOLD AND MYCOTOXIN EXPOSURE

(71) Applicant: MYCOTOXINS THERAPY, LLC, Westlake Village, CA (US)

(72) Inventor: JOSEPH BREWER, LEAWOOD, KS (US)

(73) Assignee: MYCOTOXINS THERAPY, LLC, Westlake Village, CA (US)

(21) Appl. No.: 14/827,249

(22) Filed: Aug. 14, 2015

Related U.S. Application Data
(60) Provisional application No. 62/094,830, filed on Dec. 19, 2014.

(51) Int. Cl. A61K 9/00; A61M 11/00; A61K 45/06; A61M 31/00; A61M 3/02; A61K 31/7048; A61M 16/06; A61M 15/08

(52) U.S. Cl. CPC A61K 9/0043; A61M 16/0666; A61M 11/00; A61M 15/08; A61M 31/00; A61M 3/0279; A61K 31/7048; A61K 45/06; A61M 2210/0618; A61M 2210/0681

(57) ABSTRACT

A method of treating disease states associated with mold and mycotoxin , resulting in illnesses such as Chronic Fatigue Syndrome (CFS), Gulf War Syndrome (GWS), fungal sinusitis, Rhino Sinusitis, Chronic Rhino Sinusitis (CRS), abnormalities in T and B cells, central and peripheral nervous system disorders, asthma, sarcoidosis, respiratory infections, fibromyalgia, headache, depression, autoimmune disorders, and cancer, among others, comprising delivery of a medication to the nasal passages and/or sinus cavities of a patient, wherein the medication comprises an antifungal agent.

### CROSS-REFERENCE TO RELATED APPLICATIONS

This application claims the benefit of U.S. Provisional Patent Application Ser. No. 62/094,830, filed Dec. 19, 2014, and which is hereby incorporated herein by reference in its entirety.

### TECHNICAL FIELD

The disclosed technology relates generally to the treatment of acute and chronic illnesses caused by, or suspected to be caused by, fungus, mold, and/or gram-negative and gram-positive bacteria, and more particularly, some embodiments relate to the use of topical solutions, sprays, mists, powders and aerosols used to treat fungus, mold, and/or gram-negative and gram-positive bacteria within the nasal passages and sinus cavities.

### DESCRIPTION OF THE RELATED ART

Mycotoxins are a diverse group of low molecular-weight molecules produced as secondary metabolites of filamentous fungi. There are several different groups of mycotoxins, including aflatoxins, ochratoxins, gliotoxin, ergot alkaloids, trichothecenes, and many others. Each of these mycotoxins may be produced by more than one type of mold or fungus, and each mold or fungus may also produce more than one type of mycotoxin.

Research over the past 25 years has shown the adverse health effects of persons exposed to mycotoxins and mycotoxin-producing molds. Mycotoxins have been shown to suppress all aspects of the immune system and result in acute and chronic illnesses. Mycotoxins may enter the body because of external factors, resulting in acute exposure, ongoing exposure, or re-exposure to mold and mycotoxin-producing organisms.

Chronic Fatigue Syndrome (CFS), also called myalgic encephalitis, has been widely studied and numerous mechanisms and theories have been proposed to explain its pathophysiology, epidemiology, and causation. CFS patients have demonstrated evidence of increased viral activation, oxidative stress, immune abnormalities, neurocognitive features, endocrine abnormalities, impaired oxidative phosphorylation, low adenosine triphosphate (ATP) production within cells, and increased lactic acid with exercise. Symptoms of CFS include, but are not limited to, fatigue, muscle and/or joint aching, headaches, loss of balance, neurocognitive difficulties, flu-like symptoms, irritable bowel syndrome, anxiety, depression, and others.

Many patients with CFS have mitochondrial dysfunction, and thus have similarities to mitochondrial diseases. Given that all human cells contain mitochondria, mitochondrial dysfunction may affect all cells including the brain, heart, liver, skeletal muscles, kidneys, the endocrine system, and the respiratory system. Mycotoxins can lead to mitochondrial dysfunction as a result of several mechanisms.

Manifestations of mold and mycotoxin illness can include autoimmune disorders, chronic fatigue, neurodegenerative disorders (such as amyotrophic lateral sclerosis, multiple sclerosis, and Parkinson's disease), depression, other psychiatric disorders, hormone imbalance, cancer, and premature aging, among others.

Concurrent diagnosis of CFS associated with Sick Building Syndrome (SBS) has been reported. One known cause of SBS is the presence of mold within the building. Water-damaged buildings (WDB) and other damp environments suitable for fungal growth contain a complex mixture of biocontaminants produced by both mold and bacteria. Mycotoxins have been detected in the dust, carpeting, wallpaper, heating, ventilation and air-conditioning (HVAC) systems, and respirable airborne particulates of WDB. Persons exposed to WDB have experienced chronic illnesses associated with mycotoxin exposure and have been shown to have the presence of mycotoxins in their urine and within their body. Persons exposed to WDB frequently exhibit clinical features similar to CFS. Studies have demonstrated that mycotoxins may be an underlying cause of mitochondrial dysfunction in persons exposed to molds, such as those found in WDB.

Treatment of diseases such as CFS has generally focused on relief of symptoms. However, symptoms recur often and are only partially controlled, if at all. These treatments fail to consider potential underlying causes for symptom recurrence and accentuation.

### BRIEF SUMMARY OF EMBODIMENTS

According to various embodiments of the disclosed technology, a treatment of illnesses caused by or suspected to be caused by the presence of fungus, mold, and/or gram-negative and gram-positive bacteria, and subsequent byproducts and/or components of fungus, mold, and/or gram-negative and gram-positive bacteria located within the nasal passages and sinus cavities is provided.

According to various embodiments of the disclosed technology, a method of treating disease states associated with mold and mycotoxins is presented, the method comprising delivering a medication to one or more nasal passages and sinuses cavities of a patient. In various embodiments, the medication comprises an antifungal agent, an antifungal agent and one or more antibiotics, and an antifungal agent and one or more biofilm inhibitors. Other embodiments may include leukotriene antagonists, mucolytics, and possibly other related mold, fungal, bacterial biofilm inhibitors. Delivering the medication may include, in various embodiments, use of a liquid medication administration device, such as a nebulizer, atomizer, or spray bottle, or a syringe when the medication is a medicated gel.

Other features and aspects of the disclosed technology will become apparent from the following detailed description, taken in conjunction with the accompanying drawings, which illustrate, by way of example, the features in accordance with embodiments of the disclosed technology. The summary is not intended to limit the scope of any inventions described herein, which are defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof.

FIG. 1 is an example flow diagram of one method of treating disease states associated with mold and mycotoxin exposure in accordance with the technology of the present disclosure.

FIG. 2 is another example flow diagram of one method of treating disease states associated with mold and mycotoxin exposure in accordance with the technology of the present disclosure.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION OF THE EMBODIMENTS

Embodiments of the technology disclosed herein are directed toward treatment of illnesses caused by, or suspected to be caused by, the presence of fungus, mold, and/or gram-negative and gram-positive bacteria, and subsequent byproducts and/or components of fungus, mold, and/or gram-negative and gram-positive bacteria located within the nasal passages and sinus cavities. More particularly, the various embodiments of the technology disclosed herein relate to topical solutions, sprays, mists, powders, and aerosols that are used to treat diseases suspected to be caused by mycotoxins produced by mold, fungus, and bacteria within the nasal passages and cavities. Disease states include but are not limited to: Chronic Fatigue Syndrome (CFS), Gulf War Syndrome (GWS), fungal sinusitis, Rhino sinusitis, Chronic Rhino sinusitis (CRS), abnormalities in T and B cells, central and peripheral nervous system disorders (Alzheimer's disease, Parkinson's disease, etc.), asthma, sarcoidosis, respiratory infections, fibromyalgia, headache, depression, autoimmune disorders, hormone imbalances, pre-mature aging and cancer, among others.

In a study looking at the connection between patients with CFS and exposure to WDB, the inventors discovered the presence of mycotoxins within patients suffering from CFS. Of the 112 patients meeting the criteria for CFS, 90% admitted to having prior exposure to WDB. Further discussion of the scientific method employed to conduct the study may be found in “Detection of Mycotoxins in Patients with Chronic Fatigue Syndrome,” the disclosure of which is herein incorporated by reference. It is hypothesized that the continued presence of mycotoxins in the patients resulted in and accentuated the symptoms of CFS. However, many of the patients' exposure to WDB and mycotoxin-producing organisms were remote, some having not had any exposure to WDB for years prior to the detection of mycotoxins within their bodies. The presence of high levels of mycotoxins within the patients indicates some continued source of exposure to mycotoxins, whether from an internal source, an external source, or both.

The inventors have discovered that exposure to mold, mold contaminants, and mycotoxins can lead to chronic illness as a result of mold byproducts being harbored internally, within the sinus cavities, and continually producing and releasing mycotoxins within the human body. Details regarding the finding of a link between mycotoxin exposure and illnesses evidencing similar symptoms to mycotoxin exposure, such as CFS, may be found in “Chronic Illness Associated with Mold and Mycotoxins: Is Naso-Sinus Fungal Biofilm the Culprit?”, the disclosure of which is herein incorporated by reference.

The nasal sinuses have been shown to virtually always harbor fungal species, including those that have the potential to produce mycotoxins, such as aspergillus, chaetomium, fusarium, penicillium, and trichoderma, among others. Nasal washings may remove some of the mold and mold contaminants harbored in the nasal passages, but mycotoxin-related illnesses and symptoms persist in many patients.

The lack of success merely flushing the sinuses has had with eliminating the recurrence of mycotoxin associated illnesses and symptoms may be associated with the biofilm created by many molds and bacteria. Biofilms are complex surface-associated populations of microorganisms embedded in an extracellular matrix (ECM) that possess distinct phenotypes compared to planktonic (free living) organisms. The ECM is a slime-like substance that exerts a stronger adhesion to a surface as the biofilm develops, until the biofilm has reached a state of irreversible attachment. Once irreversibly attached, the mold, fungus, and other microorganisms continue to mature, resulting in the ongoing production and release of mycotoxins.

Biofilms confer considerable protection for the organisms residing inside, including resistance to host defenses and antibiotic and antifungal treatments. The ECM acts as a physical barrier, protecting the fungal cells and microorganisms within the biofilm from the clinically useful antibiotic and antifungal agents. This allows the fungal cells and microorganisms to continue to thrive and produce secondary metabolites, such as mycotoxins. As long as the biofilms are present within the nasal cavities, the fungal cells and microorganisms are protected and may continue to produce mycotoxins. By utilizing an antifungal agent, and possibly combining the antifungal agent with an antibiotic, EDTA, polysorbate 80, or other biofilm disrupter, a more effective treatment may be possible that would remove the source of the continued mycotoxin production within the body.

Through treating the mycotoxin-producing reservoir within the nasal passages and sinus cavities, the recurrence and relative strength of symptoms related to CFS, GWS, fungal sinusitis, abnormalities in T and B cells, central and peripheral nervous system disorders, asthma, sarcoidosis, respiratory infections, fibromyalgia, headache, depression, autoimmune disorders, hormone imbalances, pre-mature aging, cancer, and other disease states may be more effectively treated. Attacking the internal source would eliminate the continued production and exposure to mycotoxins that accentuate the symptoms of the disease states. This treatment goes beyond merely treating the symptoms of the disease and seeks to attack the underlying source of mycotoxin production and disease manifestation.

In a pilot study, 151 patients who tested positive for the presence of mycotoxins were treated with a medicated compound in accordance with the treatment disclosed herein. Each patient was treated with the medication once per day and for a period of greater than six months. Of the 94 patients who continued the therapy, 94% of those patients achieved clinical improvement of their symptoms. Clinical improvement was measured as improvement of 25-50% or greater in the potency of the symptoms. In the patients that had improved with the treatment disclosed herein, it was also found that the mycotoxin levels in the urine decreased markedly from baseline levels. These results evidence the increased efficacy of treating chronic illnesses, such as CFS, as well as other mycotoxin related illnesses noted above.

Non-limiting examples of antibiotics include the following classes of products: cephalosporins (1^{st}-4^{th} generation), penicillins, aminoglycosides, quinolones, tetracyclines, and macrolides. Non-limiting examples of antifungals include: amphotericin B, fluconazole, itraconazole, nystatin, micafungin, caspofungin, and all forms of liposomal amphotericin. Non-limiting examples of leukotriene antagonists include: montelukast and zafirlukast. Non-limiting examples of mucolytics include: acetylecysteiene, dornase alpha, and saline. Non-limiting examples of biofilm inhibitors include: EDTA, polysorbate 80, mupirocin, and other bactericidal agents.

In addition to the antifungals, antibiotics, leukotriene antagonists, mucolytics, and biofilm inhibitors discussed above, various embodiments may include additional compounds, including anti-inflammatories, antihistamines, decongestants, antivirals, and antimicrobial medications.

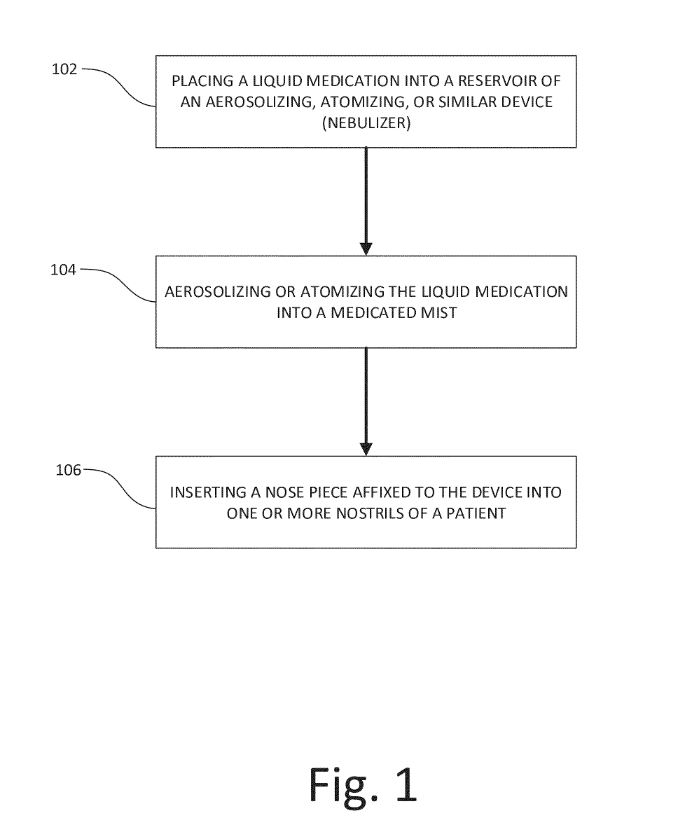

One method of treating illnesses suspected to be caused by mycotoxins produced by mold, fungus, and bacteria within the nasal passages and sinus cavities is through aerosolized- or atomized-administration of the medication. Embodiments using this method involve the use of an aerosolizer, such as a nebulizer, or an atomizer to aerosolize a liquid medication for topical administration through the patient's sinus passages. Various embodiments utilizing this method of treatment may include one or more of the following, as shown in the example flow diagram of FIG. 1: 1) liquid medication ranging from 0.5 ml-10 ml placed in the reservoir of an aerosolizing, atomizing, or similar device at 102; 2) aerosolizing or atomizing the liquid into particle sizes ranging from 0.1 μm-99 μm or larger at 104, creating a medicated mist; and 3) inserting a nose piece affixed to the device into the nostril(s) to administer the aerosolized or atomized medication for a period ranging from 0.5 min-30 min or longer at 106.

Another method of treating illnesses suspected to be caused by mycotoxins produced by mold, fungus, and bacteria within the sinus passages and nasal cavities is through use of a nasal spray. A spray device includes a spray nozzle that transforms the liquid medication into a medicated mist for application to the nasal passages and sinus cavities. Various embodiments utilizing this method of treatment may include one or more of the following: 1) a liquid medication ranging from 0.1 ml-10 ml placed into a nasal spray bottle adequately suited for nasal use; and 2) administering the liquid medication into the nasal passages of each nostril via the spray bottle using 1-10 sprays per nostril. In various embodiments, the liquid medication may be diluted with 0.5 ml-20 ml saline or other diluents.

The liquid medication may be created by mixing 2 mg-50 mg of an antifungal, antibiotic, leukotriene antagonist, mucolytic, or a biofilm inhibitor, or a combination thereof, in a 0.1 ml-10 ml solution. In various embodiments, the liquid medication may also include one or more of anti-inflammatories, antihistamines, decongestants, antivirals, or antimicrobial medications.

Another method of treating illnesses suspected to be caused by mycotoxins produced by mold, fungus, and bacteria within the nasal passages and sinus cavities is through nasal rinsing or irrigation. Generally, nasal rinses and irrigation systems are used to flush out excess mucus and debris from the nasal passages and sinus cavities, but they can also be used to administer medicated solutions to the nasal passages and sinus cavities. Various embodiments utilizing this method of treatment may include one or more of the following: 1) a liquid medication ranging from 0.1 ml-10 ml added to a commercially available or prepared nasal rinsing or irrigation solution; and 2) administering the liquid medication to the nasal passages and sinus cavities by rinsing, flushing, irrigating, or otherwise exposing nasal passages and cavities to the combined medication and rinsing or irrigating solution. Examples of commercially available nasal rinsing or irrigation solutions include but are not limited to ASL Pharmacy's ActiveSinus saline rinse, Neilmed Nasal Rinses, and other solutions.

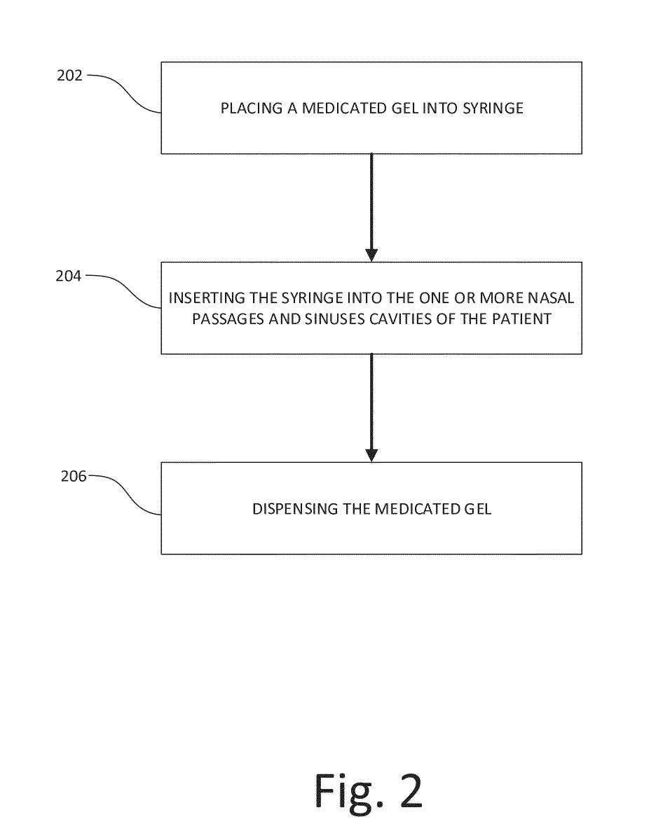

Another method of treating illnesses suspected to be caused by mycotoxins produced by mold, fungus, and bacteria within the nasal passages and sinus cavities is through the use of a medicated gel. The medicated gel may comprise a viscous fluid having a viscosity sufficient to be maintained within a human sinus cavity after insertion and a medication dispersed within the viscous fluid. Various embodiments utilizing this method of treatment may include one or more of the following, as shown in the example flow diagram of FIG. 2: 1) a syringe filled with a medicated gel at 202; and 2) administering the medicated gel to the nasal passages by inserting the syringe into the nostril at 204 and dispensing the medicated gel into the sinus cavity at by depressing the plunger of the syringe at 206.

In various embodiments, topical solutions, sprays, mists, powders, and aerosols, as do the compositions from which they arise, may include one or more compounds selected from the following class of compounds: antibiotics, antifungals, leukotriene antagonists, mucolytics, and other related mold, fungal, bacterial biofilm inhibitors. It is noted that in many embodiments, a single antifungal, antibiotic, leukotriene antagonist, mucolytic, or other related mold, fungal, bacterial biofilm inhibitor compound may be used. The following are non-limiting examples where more than one compound is included: one antibiotic and one antifungal; one antibiotic, one antifungal, and one biofilm inhibitor; one antibiotic, one antifungal, and one mucolytic; one antifungal, one mucolytic and one biofilm inhibitor; one antifungal and one biofilm inhibitor, among others.

The time of a single administration of a topical solution, spray, mist, powder, or aerosol typically varies from 0.5 min-30 min in length or longer, depending on the composition of the compound and the administration device and/or process. Treatment may involve 1-4 administrations per day and treatment duration may last from 7 days to 1 year, or longer depending on the extent of the illness. Topical solutions, sprays, mists, powders, and aerosols of the technology herein disclosed may also be used as a prophylactic treatment.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A method comprising:
delivering a medication to one or more nasal passages and sinuses cavities of a patient, wherein the medication comprises an antifungal agent.

2. The method of claim 1, wherein the antifungal agent comprises one or more of:
amphotericin B, fluconazole, itraconazole, nystatin, micafungin, or caspofungin.

3. The method of claim 1, wherein the antifungal agent comprises nystatin.

4. The method of claim 1, the medication further comprising a biofilm inhibitor, the biofilm inhibitor comprising one or more of: EDTA, polysorbate 80, or mupirocin.

5. The method of claim 1, wherein the medication is a liquid medication and delivering the medication comprises:
placing the medication in a reservoir of a liquid medication administration device; and
administering, via the liquid medication administration device, the medication to the one or more nasal passages and sinuses cavities of the patient.

6. The method of claim 5, wherein the liquid medication administration device is a nebulizer or atomizer, and administering the medication comprises inserting a nose piece affixed to the nebulizer or atomizer in the one or more nasal passages and sinuses cavities of the patient.

7. The method of claim 5, wherein the liquid medication administration device is a spray bottle.

8. The method of claim 5, wherein the liquid medication administration device is a nasal irrigation device.

9. The method of claim 1, wherein the medication is a gel, and delivering the medication comprises:
placing the medication into a syringe;
inserting the syringe into the one or more nasal passages and sinuses cavities of the patient; and
dispensing the medicated gel by depressing a plunger of the syringe.

10. The method of claim 1, further comprising delivering a chelating agent to one or more nasal passages and sinus cavities of a patient, the chelating agent comprising a biofilm inhibitor, wherein delivering the chelating agent occurs before delivering the medication.

11. The method of claim 10, wherein the biofilm inhibitor comprises one or more of: EDTA, polysorbate 80, or mupirocin.

12. The method of claim 1, the medication further comprising one or more of: an antibiotic; a mucolytic; or a leukotriene antagonist.

13. The method of claim 12, the antibiotic comprising one or more of: cephalosporin (1^{st}-4^{th} generation); penicillin; aminoglycoside; quinolone; tetracycline; or macrolide.

14. The method of claim 12, the mucolytic comprising one or more of:
acetylecysteiene; dornase alpha; or saline.

15. The method of claim 12, the leukotriene antagonists comprising one or more of montelukast or zafirlukast.

16. The method of claim 1, the medication further comprising one or more of: anti-inflammatory; antihistamine; decongestant; antiviral; or antimicrobial.

17. The method of claim 1, wherein the method is directed to treating disease states associated with mold and mycotoxins, the disease states associated with mold and mycotoxins comprising one or more of: Chronic Fatigue Syndrome (CFS); Gulf War Syndrome (GWS); fungal sinusitis; Rhino sinusitis; Chronic Rhino Sinusitis (CRS); abnormalities in T and B cells; central and peripheral nervous system disorders; asthma; sarcoidosis; respiratory infections; fibromyalgia; headache; depression; autoimmune disorders; hormone imbalances; pre-mature aging; or cancer.

18. A method comprising:
placing a liquid medication in a reservoir of a nebulizer;
inserting a nose piece affixed to the nebulizer or atomizer in one or more nasal passages and sinuses cavities of a patient; and
delivering the medication to the one or more nasal passages and sinuses cavities of the patient;
wherein the liquid medication comprises nystatin.

19. A method comprising:
placing a liquid medication in a reservoir of an atomizer;
inserting a nose piece affixed to the atomizer in one or more nasal passages and sinuses cavities of a patient; and
delivering the medication to the one or more nasal passages and sinuses cavities of the patient;
wherein the liquid medication comprises nystatin.

20. A medicated mist prepared by a process comprising the steps of:
placing a liquid medication in a reservoir of a nebulizer;
inserting a nose piece affixed to the nebulizer in one or more nasal passages and sinuses cavities of a patient; and
delivering the liquid medication to the one or more nasal passages and sinuses cavities of the patient;
wherein the liquid medication comprises nystatin.
